(19) United States
(12) Patent Application Publication
(10) Pub. No.: US 2020/0215169 A1
(43) Pub. Date: Jul. 9, 2020

(54) ANTIBACTERIAL METHODS AND RELATED KITS

(71) Applicant: THE HENRY M. JACKSON FOUNDATION FOR THE ADVANCEMENT OF MILITARY MEDICINE, INC., BETHESDA, MD (US)

(72) Inventors: Kristi Lynn FRANK, Silver Spring, MD (US); Candace ROUCHON, Washington, DC, DC (US); Joann Arathi HARRIS, Laurel, MD (US)

(21) Appl. No.: 16/631,225

(22) PCT Filed: Jul. 17, 2018
(86) PCT No.: PCT/US2018/042447

§ 371 Date: Jan. 15, 2020

Related U.S. Application Data
(60) Provisional application No. 62/533,445, filed on Jul. 17, 2017.

(51) Int. Cl. A61K 38/47; A61K 45/06; A61K 9/00; A61P 31/04; C12Q 1/18

(52) U.S. Cl. CPC A61K 38/47; C12Y 302/01017; A61K 45/06; C12Q 1/18; A61K 9/0024; A61P 31/04; A61K 9/0048

(57) ABSTRACT

Methods of treating a bacterial infection using lysozyme are provided. Related methods of preventing bacterial growth and monitoring bacterial growth are also provided. Kits are also provided.

### GOVERNMENT INTEREST

This invention was made with government support under grant R0733973 by the Uniformed Services University. The government has certain rights in the invention.

### FIELD OF THE INVENTION

The invention relates generally to infectious agents and more specifically to treating or preventing bacterial infections in certain embodiments.

### BACKGROUND OF THE INVENTION

Bacterial biofilms, a form of growth in which bacteria attach to and grow on surfaces, are involved in the majority of bacterial infections. They are very difficult infections to treat because the bacteria can become resistant to very high concentrations of antibiotics upon attaching to a surface. Bacteria form biofilms on biotic and abiotic surfaces throughout nature, the built environment, and in hosts. For example, bacterial biofilms can form directly on surfaces in a patient, such as heart valves or wounds, and on any type of implanted medical device, including intravenous and urinary catheters, and orthopedic implants and hardware.

Biofilms are highly ordered microbial populations of cells attached to a surface and to each other. Biofilm-associated bacteria display phenotypes disparate from those of planktonic bacteria, which are defined as free-floating or non-attached cells. Formation of a biofilm provides protection from adverse environmental conditions, such as nutrient deprivation, desiccation, and opsonization and phagocytosis by host immune systems. Individual cells within biofilms display differential patterns of gene expression within distinct areas of the microbial community.

Biofilm cellular populations adopt a basic architecture comprising microcolonies interspersed with channels for fluid exchange. One characteristic of a biofilm is self-production of a polymeric extracellular matrix (ECM), which provides protective and structural support to the overall architecture of the microbial community. The ECM may be composed of polysaccharides, proteins, and extracellular nucleic acids. Biofilms are inherently resistant to antimicrobial agents. For example, antibiotic concentrations up to one thousand times higher than those that inhibit planktonic cells may be necessary to elicit inhibitory effects on biofilm cells. Treatment options are limited for biofilm-associated bacterial infections, making them a major threat to human health.

Enterococcus faecalis, for example, is a bacterium that frequently exhibits antibiotic resistance and causes several types of infections that involve biofilm formation. E. faecalis is a Gram-positive bacterium that inhabits the gastrointestinal tract of humans and other animals as a commensal. E. faecalis is also an opportunistic pathogen that is a leading cause of healthcare-associated infections. The clinical significance of E. faecalis infection derives, in part, from the organism's innate and acquired resistance to many antibiotics and its ability to form biofilms. For example, E. faecalis biofilms are frequently found in secondary endodontic infections, infective endocarditis, post-surgical endophthalmitis, catheter-associated bloodstream infections, catheter-associated urinary tract infections, and wound infections (often polymicrobial), as well as on intravenous catheter tubing and implanted orthopedic hardware.

In view of the foregoing, it is apparent that improved approaches to treat and prevent bacterial infections, such as those which exhibit antibiotic resistance and grow in biofilms, are needed.

### SUMMARY OF THE INVENTION

The present disclosure is based, in part, on the surprising discovery that exposing biofilms (e.g., those formed by E. faecalis) to lysozyme formulations reduces the number of viable bacterial cells in those biofilms. This is an unexpected result because, prior to the present disclosure, it was established that planktonic E. faecalis cells become resistant to lysozyme following exposure to it. Lysozyme is an enzyme found in mammalian immune cells and mucosal secretions that hydrolyzes bonds between the subunits that form bacterial cell walls. This exposure or contact to lysozyme can be at the site of an infection where a biofilm has formed, whether on an external surface wound of a subject or internal to a subject, as with a urinary tract infection. Accordingly, the present disclosure provides methods, kits, and compositions for treating bacterial infections, as well as preventing and monitoring bacterial growth. The methods, kits, and compositions generally include using effective amounts of lysozyme to reduce the number of bacterial organisms at the site of an infection or contamination, for example where a biofilm has formed.

In one aspect, for example, the disclosure provides a method of treating a bacterial infection associated with a biofilm. The method includes administering a therapeutically effective amount of lysozyme to a subject that is infected with bacteria that produce the biofilm in and/or on the subject. The lysozyme is typically exogenous to the subject. In certain embodiments, for example, the lysozyme administered to the subject is obtained from chicken egg white or is a recombinant human lysozyme. In certain embodiments, the etiologic agent of the bacterial infection is Enterococcus faecalis. In other embodiments, other biofilm-forming bacterial organisms are targeted using these methods. In some embodiments, the subject is a mammalian subject (e.g., a human subject, a non-human mammalian subject, etc.). In certain embodiments, the method also includes administering a therapeutically effective amount of an antibacterial agent (e.g., penicillin, imipenem, vancomycin, daptomycin, linezolid tedizolid, tigecycline, etc.) or a pharmaceutically acceptable salt thereof to the subject. In some embodiments, the method includes topically administering the therapeutically effective amount of the lysozyme to the subject (e.g., to a wound, to an eye, or the like).

In other embodiments, the biofilm is on a medical device (e.g., a stent, a catheter (such as, a urinary tract catheter, an intravenous catheter, etc.), a pacemaker, a prosthetic joint, a prosthetic heart valve, etc.) or exogenous biological component (e.g., an animal or cadaver heart valve, etc.) before and/or after that device or component is implanted in the subject. In some embodiments, for example, medical devices and exogenous biological components are treated with lysozyme formulations to remove potential bacterial biofilms prior to being implanted in subjects. In other exemplary embodiments, lysozyme formulations are administered to subjects post-implantation to treat bacterial infections in those subjects. Typically, the methods include administering the therapeutically effective amount of the lysozyme in a solution that comprises a concentration of the lysozyme between about 0.1 mg/ml and about 10.0 mg/ml (e.g., between about 0.15 mg/ml and about 5.0 mg/ml, between about 1.25 mg/ml and about 2.5 mg/ml, etc.). In some embodiments, the method includes administering the therapeutically effective amount of the lysozyme to the subject for between about three hours and about 24 hours.

In another aspect, the disclosure provides a method of monitoring bacterial growth. The method includes contacting a sample that includes a population of target bacterial organisms that produces a biofilm with a solution that comprises an antibacterial effective amount of lysozyme, such as between about 0.1 mg/ml and about 10.0 mg/ml for between about three hours and about 24 hours. The method also includes detecting at least one property of the population of target bacterial organisms indicative of bacterial growth prior to, during, and/or after the contacting step, thereby monitoring the bacterial growth. In some embodiments, the property comprises an amount of biomass in the population of target bacterial organisms in the sample. In certain embodiments, the sample is from a mammalian subject. In some embodiments, the population of target bacterial organisms comprise Enterococcus faecalis. Optionally, the concentration of the lysozyme is between about 0.15 mg/ml and about 5.0 mg/ml. In certain embodiments, the concentration of the lysozyme is about 1.25 mg/ml. In some embodiments, the sample and the solution are contacted for between about three hours and about 24 hours.

In another aspect, the disclosure provides a kit that includes (a) a medical device or an exogenous biological component (e.g., a stent, a catheter, a pacemaker, a prosthetic joint or other orthopedic implants, a prosthetic heart valve, an animal heart valve, a cadaver heart valve, etc.), and (b) a container comprising a solution that comprises an antibacterial concentration of lysozyme. In some embodiments, the container includes the medical device or the exogenous biological component (e.g., stored in the solution to prevent bacterial biofilm formation).

In another aspect, the disclosure provides a kit that includes a medical device that contains a solution that comprises an antibacterial concentration of lysozyme (e.g., a catheter filled with the solution or the like).

### BRIEF DESCRIPTION OF THE DRAWINGS

The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1A shows a graph of results from quantified biofilm biomass measurements obtained by reading the optical density (shown on the y-axis) of respective safranin-stained cultures of two Enterococcus faecalis strains (OG1RF (including Eep protease) and Δeep (lacking Eep protease) strains; shown on the x-axis) in the wells of 96-well polystyrene plates at OD_{450 nm} after being treated with a lysozyme (hen egg white lysozyme) solution or a buffer solution lacking lysozyme.

FIG. 1B shows a graph of results from measurements of the number of viable cells (colony forming units (CFU); shown on the y-axis) recovered from these biofilms.

FIG. 1C shows a graph of results from quantified biofilm biomass measurements obtained by reading the optical density (shown on the y-axis) of respective safranin-stained cultures of two E. faecalis strains (OG1RF and Δeep strains; shown on the x-axis) in the wells of 96-well polystyrene plates at OD_{450 nm} after being treated with an ampicillin solution or a buffer solution lacking ampicillin.

FIG. 1D shows a graph of results from measurements of the number of viable cells (CFU; shown on the y-axis) recovered from these biofilms.

FIG. 2A shows a graph of results from quantified biofilm biomass measurements obtained by reading the optical density at OD_{450 nm} (shown on the y-axis) of respective safranin-stained cultures of two E. faecalis strains (OG1RF and Δeep strains; shown on the x-axis) after being treated for various durations (3, 6, or 24 hours) with a lysozyme (hen egg white lysozyme) solution or a buffer solution lacking lysozyme.

FIG. 2B shows a graph of results from measurements of the number of viable cells (CFU; shown on the y-axis) recovered from these biofilms.

FIG. 3A shows a graph of results from quantified biofilm biomass measurements obtained by reading the optical density at OD_{450 nm} (shown on the y-axis) of respective safranin-stained cultures of two E. faecalis strains (OG1RF and Δeep strains; shown on the x-axis) after being treated with solutions having various concentrations of lysozyme (0.156 mg/ml, 1.25 mg/ml, or 5 mg/ml; hen egg white lysozyme) or a buffer solution lacking lysozyme.

FIG. 3B shows a graph of results from measurements of the number of viable cells (CFU; shown on the y-axis) recovered from these biofilms.

FIG. 4A shows a graph of results from quantified biofilm biomass measurements obtained by reading the optical density at OD_{450 nm} (shown on the y-axis) of respective safranin-stained cultures of two E. faecalis strains (OG1RF and Δeep strains; shown on the x-axis) after being treated with a lysozyme (recombinant human lysozyme) solution or a buffer solution lacking lysozyme.

FIG. 4B shows a graph of results from measurements of the number of viable cells (CFU; shown on the y-axis) recovered from these biofilms.

FIG. 5A shows a graph of results from quantified DNA measurements (in relative fluorescence units (RFU); shown on the y-axis) obtained from biofilms of two cultured E. faecalis strains (OG1RF and Δeep strains; shown on the x-axis) after being treated with a lysozyme solution or a buffer solution lacking lysozyme.

FIG. 5B shows a graph of results from measurements of the number of viable cells (CFU; shown on the y-axis) recovered from these biofilms.

FIG. 6 shows a graph of results from quantified biofilm biomass measurements obtained by reading the optical density (shown on the y-axis) of respective safranin-stained cultures of two E. faecalis strains (OG1RF and Δeep strains; shown on the x-axis) in the wells of 96-well microtiter plates at OD_{450 nm} after being treated with a lysozyme (5 mg/ml) solution or a buffer solution lacking lysozyme in which lysozyme was either added to the microtiter plates immediately following a washing step (labeled “Wet”) or after the microtiter plates were dried following the washing step (labeled “Dry”). These assays assessed the effect of allowing the microtiter plate to dry before the addition of lysozyme treatment.

FIG. 7A shows a graph of results from quantified biofilm biomass measurements obtained by reading the optical density (shown on the y-axis) of respective safranin-stained cultures of two Enterococcus faecalis strains (OG1RF and Δeep strains; shown on the x-axis) in the wells of 96-well polystyrene plates at OD_{450 nm} after being treated with a lysozyme (hen egg white lysozyme) solution or a buffer solution lacking lysozyme.

FIG. 7B shows a graph of results from measurements of the number of viable cells (colony forming units (CFU); shown on the y-axis) recovered from these biofilms or the supernatants obtained after lysozyme or buffer treatment of the strains (to measure whether cells were being dispersed from the biofilm).

FIG. 8A is a graph showing the quantification of the number of viable Δeep and OG1RF logarithmic phase cells (y-axis) following exposure to either water or a lysozyme solution over a 6-hour period (x-axis).

FIG. 8B is a graph showing the quantification of the number of viable Δeep and OG1RF stationary phase cells (y-axis) following exposure to either water or a lysozyme solution over a 6-hour period (x-axis).

FIG. 9A shows a graph of results from quantified biofilm biomass measurements obtained by reading the optical density (shown on the y-axis) of respective safranin-stained cultures of 7 Enterococcus faecalis strains (OG1RF, DS16, FA2-2, JH2-2, VA1128, V583, and 39-5 strains; shown on the x-axis) in the wells of 96-well polystyrene plates at OD_{450 nm} after being treated with a lysozyme (hen egg white lysozyme) solution or a buffer solution lacking lysozyme.

FIG. 9B shows a graph of results from measurements of the number of viable cells (colony forming units (CFU); shown on the y-axis) recovered from these biofilms.

### DETAILED DESCRIPTION

Bacterial infections that involve biofilm formation are often challenging to effectively treat. Enterococcus faecalis, for example, is a Gram-positive gastrointestinal commensal and a leading cause of nosocomial infections. E. faecalis infections are difficult to treat, in part, because the organism forms biofilms and is resistant to many antimicrobial agents. Previous studies have demonstrated that lysozyme resistance is stimulated through a signal transduction cascade that involves activation of the alternative sigma factor SigV via cleavage of the anti-sigma factor RsiV by transmembrane metalloprotease Eep. Under planktonic conditions, strains lacking the eep gene are more sensitive than wild-type strains to growth inhibition by lysozyme. Since bacteria in biofilms gain resistance to high concentrations of antimicrobials through biofilm-specific mechanisms, it was investigated whether E. faecalis OG1RF Δeep biofilms would remain differentially susceptible to lysozyme as compared to wild-type (E. faecalis OG1RF) biofilms. It was initially hypothesized that the amount of biomass in Δeep biofilms following exposure to lysozyme would be equal to or less than the biomass of OG1RF biofilms. As described further herein, it was unexpectedly found that a three-hour treatment with lysozyme was associated with increased biofilm biomass of equal magnitude for both strains and concurrent decreases in biofilm cell viability of 99.8% and 99.9% for OG1RF and Δeep, respectively. In contrast, three-hour treatment with the cell wall-targeting antibiotic ampicillin caused no changes in biofilm biomass or cell viability of either strain. LIVE/DEAD florescence staining showed a higher percentage of dead cells in lysozyme-treated OG1RF and Δeep biofilms relative to biofilms treated with buffer alone. Taken together, these results suggest that E. faecalis biofilm cells lyse following treatment with lysozyme, and the increased biofilm staining observed following lysozyme treatment may be due to the release of DNA from the lysed cells. Consistent with this, approximately 3-fold more extracellular DNA was measured in association with lysozyme-treated biofilms than with biofilms treated with buffer alone. These results demonstrate that E. faecalis biofilms are susceptible to treatment by lysozyme in a manner that is independent of Eep protease. Therefore, lysozyme has utility, for example, as a new therapeutic that can reduce the number of bacteria (e.g., E. faecalis) at the site of an infection where a biofilm has formed. Accordingly, the present disclosure provides methods, kits, and compositions for treating bacterial infections as well as preventing and monitoring bacterial growth. The methods, kits, and compositions generally include using effective amounts of lysozyme to reduce the number of bacterial organisms at the site of an infection or contamination, particularly where a biofilm has formed.

In some embodiments, compositions or formulations (including acceptable salts thereof) of lysozyme are delivered to the sites of biofilm infections that are known to be caused in whole or in part by E. faecalis or other biofilm forming bacteria to reduce the biofilm-associated bacterial burden. Such formulations may be applied topically to a subject's wound, eyes, teeth, or the like. In certain embodiments, antibacterial lysozyme formulations are present in catheter locks or flush solutions, or other medical devices. In some exemplary embodiments, lysozyme compositions are used to pre-treat implants or other medical devices prior to use with a subject to prevent or minimize the risk of bacterial infections. In some embodiments, lysozyme compositions are used as disinfectants or sanitizing agents in other medical applications as well as in household cleaning or industrial applications. To further illustrate, lysozyme compositions are also used to treat biofilms on dental instruments, or on dental implants (pre- and/or post-implantation) or directly on other surfaces in the oral cavity of a subject. In certain embodiments, lysozyme compositions are additionally used to disinfect contact lenses before and/or during use by a subject. In other embodiments, lysozyme compositions are delivered (e.g., systemically) to the site of biofilm infections that are inside a subject's body, for example, to treat endocarditis (heart valve infections) and implanted orthopedic hardware infections. Examples of delivery vehicles for the systemic delivery of lysozyme, include carbohydrate nanocapsules loaded with lysozyme (Sarkar et al. (2009), “Interfacially assembled carbohydrate nanocapsules: a hydrophilic macromolecule delivery platform,” J Biomed Nanotechnol., 5(5):456-463), lysozyme conjugated to bone-seeking aminobisphosphonate (Uludag et al. (2002), “Targeting systemically administered proteins to bone by bisphosphonate conjugation,” Biotechnol Prog., 18(3):604-611), and lipid-polymer hybrid nanoparticles loaded with lysozyme (Devrim et al. (2016), “Lysozyme-loaded lipid-polymer hybrid nanoparticles: preparation, characterization and colloidal stability evaluation,” Drug Dev Ind Pharm., 42(11):1865-1876).

Exemplary advantages of the present disclosure may include that the methods and compositions can be effective, where antibiotics are not, against biofilms. They can also allow for the reduction of antibiotic use generally, thereby limiting the spread of antibiotic resistance. In addition, the antibacterial effect of lysozyme against, for example, E. faecalis biofilms is essentially the same whether the enzyme is obtained from hen egg whites or from recombinant purified human sources. Moreover, lysozyme is a naturally occurring product already present in human or other animal species, so the risk of toxicity to these subjects is minimized.

### Definitions

It is to be understood that the present disclosure is not limited to particular methods, compositions, or kits, which can vary. As used in this specification and the appended claims, the singular forms “a,” “an,” and “the” also include plural referents unless the context clearly provides otherwise. It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In the description and claims, the following terminology, and grammatical variants thereof, will be used in accordance with the definitions set forth below.

The term “biofilm” refers to an aggregate of bacterial microorganisms in which bacterial cells adhere to each other and/or to a surface. These adherent cells are often covered with a matrix of extracellular polymeric substance (EPS), which is produced by the cells. Biofilm EPS is composed of extracellular DNA, proteins, and polysaccharides. These biofilms may form on any living or non-living surfaces, for example both on solid surfaces as colonies and on liquid surfaces as pellicles. Microbial cells growing in a biofilm are physiologically distinct from planktonic cells of the same organism.

The term “etiologic agent” refers to an organism acting as the causative agent of a disease or an abnormal physiological condition.

The “therapeutically effective amount” refers to that amount of a therapeutic agent sufficient to result in the amelioration of one or more symptoms of a disorder, prevent advancement of a disorder, cause regression of a disorder, or to enhance or improve the therapeutic effect(s) of another modality.

The “biomass” refers to the total mass of organisms or components thereof in a given area or volume.

### Lysozyme

Lysozyme (EC Number EC 3.2.1.17) (also known as muramidase or N-acetylmuramide glycanhydrolase) is an enzyme that breaks down the bacterial cell wall by catalyzing the hydrolysis of the beta-1,4-linkages between the N-acetylmuramic acid and N-acetylglucosamine subunits that form peptidoglycan, which comprises the cell wall of Gram-positive and Gram-negative bacteria. Hydrolysis of the peptidoglycan weakens the cell wall and renders the bacteria increasingly susceptible to lysis. When E. faecalis is grown under normal laboratory conditions and then exposed to lysozyme, a gene expression pathway that is dependent on Eep protease is induced, leading the organism to become resistant to high levels of lysozyme (Varahan et al. (2013), “Eep confers lysozyme resistance to Enterococcus faecalis via the activation of the extracytoplasmic function sigma factor SigV,” Journal of Bacteriology, 195(14):3125-3134.)

Lysozyme is produced by animals as part of their innate immune system. For example, lysozyme is found in mucosal secretions, including tears, and in the cytoplasmic granules of phagocytic cells. Hen egg whites contain an abundant amount of lysozyme. In humans, the lysozyme enzyme is encoded by the LYZ gene (Yoshimura et al. (1988), “Human lysozyme: sequencing of a cDNA, and expression and secretion by Saccharomyces cerevisiae,” Biochemical and Biophysical Research Communications, 150 (2):794-801.).

Additional details relating to lysozyme are also found in, for example, Blake et al. (1967), “Crystallographic studies of the activity of hen egg-white lysozyme,” Proc. R. Soc. Lond. B: Biol. Sci., 167:378-388 and Blake et al. (1967), “On the conformation of the hen egg-white lysozyme molecule,” Proc. R. Soc. Lond. B: Biol. Sci., 167:365-377. In addition, lysozyme (e.g., recombinant human lysozyme, from chicken egg white, etc.) is readily available from various commercial suppliers, including Sigma-Aldrich Co. LLC. In certain embodiments, the lysozyme is a hen egg white lysozyme. In certain embodiments, the lysozyme is a recombinant human lysozyme. Other sources of lysozyme can also be used in the methods and compositions disclosed in this application.

### Target Bacterial Organisms

The methods, compositions, and kits disclosed herein may be used to treat or monitor various types of bacterial infections. Bacterial targets generally form biofilms. In certain of these exemplary embodiments, targeted bacterial organisms are E. faecalis that have formed biofilms.

Other exemplary Gram-positive bacteria that are optionally targeted using the methods, compositions, and kits disclosed herein include those selected from staphylococci (e.g., Staphylococcus aureus (e.g., MSSA (methicillin susceptible S. aureus strains) and MRSA (methicillin resistant S. aureus), Staphylococcus coagulase-negative species (e.g., S. epidermidis, S. haemolyticus, S. lugdunensis, S. saprophyticus, S. hominis, and S. capitis)), streptococci (e.g., Streptococcus anginosus group (Streptococcus intermedius, Streptococcus anginosus, Streptococcus constellatus), Streptococcus pneumoniae, Streptobacillus moniliformis, Streptococcus pyogenes (Groups A, B, C, G, F), and Streptococcus agalactiae (Group B Streptococcus)), and Gram-positive bacilli (e.g., Actinomyces israelii, Arcanobacterium haemolyticum, Bacillus species (Bacillus anthracis, Bacillus cereus, Bacillus subtilis), Clostridium species (Clostridium difficile, Clostridium perfringens, Clostridium tetani), Corynebacterium species (Corynebacterium diphtheria, Corynebacterium jeikeium, Corynebacterium urealyticum), Erysipelothrix rhusiopathiae, Listeria monocytogenes, Lactobacillus species (e.g., L. acidophilus, L. brevis, L. buchneri, L. casei, L. fermentum, L. gallinarum, L. gasseri), Nocardia species (e.g., Nocardia asteroides, Nocardia brasiliensis), Propionibacterium acnes, and Rhodococcus equi).

Exemplary Gram-negative bacteria that are optionally targeted using the methods, compositions, and kits of disclosed herein include those selected from Gram-negative cocci and coccobacilli (Bordetella pertussis, Brucella species (Brucella abortus, Brucella canis, Brucella melitensis, Brucella suis), Eikenella corrodens, Haemophilus species (Haemophilus influenza, Haemophilus ducreyi, Haemophilus avium), Moraxella catarrhalis, Neisseria species (Neisseria gonorrhoeae, Neisseria meningitides), and Pasteurella multocida), Gram-negative bacilli, non-fermenting Gram-negative bacilli (Acinetobacter baumannii, Achromobacter xylosoxidans, Bordetella pertussis, Burkholderia species (Burkholderia cepacia, Burkholderia pseudomallei), Elizabethkingia meningoseptica, Moraxella catarrhalis, Pseudomonas aeruginosa, and Stenotrophomonas maltophilia), and anaerobic Gram-negative bacilli (e.g., Bacteroides fragilis, Bacteroides melaninogenicus, and Fusobacterium necrophorum)), Enterobacteriaceae (e.g., Escherichia coli, Klebsiella species, Salmonella species, Serratia species, etc.).

### Kits and Methods of Treatment, Prevention, and Monitoring

Various methods for treating or preventing infections caused by biofilm-forming bacteria are provided. Also provided are methods of monitoring the growth of these types of bacteria, for example, to assess effectiveness of the course of treatment of an infected subject (e.g., a human or non-human animal). The methods generally involve administering therapeutically effective amounts of exogenous lysozyme to infected subjects. In some of these embodiments, therapeutically effective amounts of the lysozyme are administered in solutions that include a concentration of the lysozyme between about 0.1 mg/ml and about 10.0 mg/ml (e.g., between about 0.15 mg/ml and about 5.0 mg/ml, between about 1.25 mg/ml and about 2.5 mg/ml, etc.). The lysozyme compositions are may be packaged as kits having varied configurations.

The methods disclosed herein may be used for the treatment, prevention, and/or monitoring of infections caused by Gram-negative and/or Gram-positive bacteria associated with bacterial biofilms. Optionally, these methods are applied to infections of the skin, soft tissues, the respiratory system, the lung, the digestive tract, the eye, the ear, the teeth, the nasopharynx, the mouth, the bones, the vagina, burn wounds, wounds related to bacteremia/septicemia, and/or endocarditis. The dosage and route of administration used in a method of treatment or prophylaxis disclosed herein depends on the specific disease/site of infection to be treated. To illustrate, the route of administration may be, for example, oral, topical, nasopharyngeal, parenteral, inhalational, intravenous, intramuscular, intrathecal, intraspinal, endobronchial, intrapulmonal, intraosseous, intracardial, intraarticular, rectal, vaginal or any other route of administration.

In some embodiments, compositions used in applications of the methods disclosed herein include formulations that protect active compounds (e.g., lysozyme, antibiotic agents, etc.) from environmental influences (e.g., proteases, oxidative reagents, immune responses, etc.) until those active compounds reach the site of infection. To illustrate, the formulations may include a capsule, pill, powder, suppository, emulsion, suspension, gel, lotion, cream, salve, injectable solution, syrup, spray, inhalant or any other medically accepted galenic formulation. Some of these formulations include suitable carriers, stabilizers, flavorings, buffers or other suitable reagents. For topical applications, formulations are optionally in the form of a lotion, cream, gel, salve or plaster. For nasopharyngeal applications, formulations may include saline solutions sprayed into nasal passages.

In some embodiments, the lysozyme compositions are administered in combination or in addition to antibiotics depending on the specific etiologic agent(s) involved in the particular infection. For example, one or more of the following antibiotics may be administered in combination with the lysozyme composition: streptomycin, tetracycline, cephalothin, gentamicin, cefotaxime, cephalosporin, ceftazidime, imipenem, 3-lactams, aminoglycosides, fluoroquinolones, macrolides, novobiocin, rifampicin, oxazolidinones, fusidic acid, mupirocin, pleuromutilins, daptomycin, vancomycin, sulfonamides, chloramphenicol, trimethoprim, fosfomycin, cycloserine, polymyxin, and the like.

In other exemplary embodiments, the methods include using lysozyme compositions to eliminate, reduce, or prevent bacterial biofilm formation on various medical devices and implants (artificial or biological), such as intravenous catheters, stents, urinary catheters, peritoneal dialysis catheters, endoscopes, dental devices, dialysis equipment, pacemaker, endotracheal tubes, voice prostheses, cerebrospinal fluid shunts, artificial heart valves, and joint prostheses, among many other examples. In some embodiments, these medical devices or implants are packaged as components of kits. In certain embodiments, these kits include containers comprising antibacterial lysozyme formulations that are separate from the medical devices or implants. In other embodiments, all or a portion of the medical devices or implants are packaged in contact with antibacterial lysozyme formulations in the containers. Kits also may be packaged with suitable instructions to guide usage of the antibacterial lysozyme formulations and/or the medical devices or implants.

### EXAMPLES

Unless indicated otherwise in these Examples, the methods involving commercial kits were done following the instructions of the manufacturers.

### Example 1

### Experimental Overview

Enterococcus faecalis, a commensal of the human gastrointestinal tract, has been found to cause many nosocomial infections. Using biofilms, E. faecalis is able to enhance its pathogenicity through the transcription of different genes. It is not known how biofilm formation affects lysozyme's interaction with E. faecalis. This study investigated the effect of lysozyme on E. faecalis biofilms formed by the E. faecalis strains OG1RF and OG1RFΔeep. The OG1RFΔeep strain lacks the eep gene, which encodes an Eep protease. When E. faecalis is grown under normal laboratory conditions and then exposed to lysozyme, a gene expression pathway that is dependent on Eep protease is induced, leading the organism to become resistant to high levels of lysozyme (Varahan et al. (2013)).

The biofilms formed by each strain were tested in different conditions to determine optimal conditions for lysozyme interaction with E. faecalis. A range of lysozyme concentrations (0.15625 mg/ml-5 mg/ml) were also tested against the strains. As discussed below, this study unexpectedly shows that lysozyme kills both OG1RF and Δeep biofilm cells equally well. These results suggest that lysozyme may be an effective treatment for biofilm-associated diseases caused by bacteria, such as E. faecalis.

### Three-Hour Lysozyme Treatment

Since bacteria in biofilms become resistant to high concentrations of antimicrobials, it was investigated whether biofilms of two E. faecalis strains, one containing Eep protease (OG1RF) and the other lacking Eep protease (Δeep) cells, would show different susceptibilities to lysozyme treatment. It was hypothesized that the amount of biomass in Δeep biofilms after treatment with lysozyme would be equal to or less than the biomass of OG1RF biofilms. This is because OG1RFΔeep, which lacks an Eep protease, was expected to have increased susceptibility to lysozyme as compared to OG1RF, and increased susceptibility to lysozyme would in turn decrease cell growth and the amount of biomass.

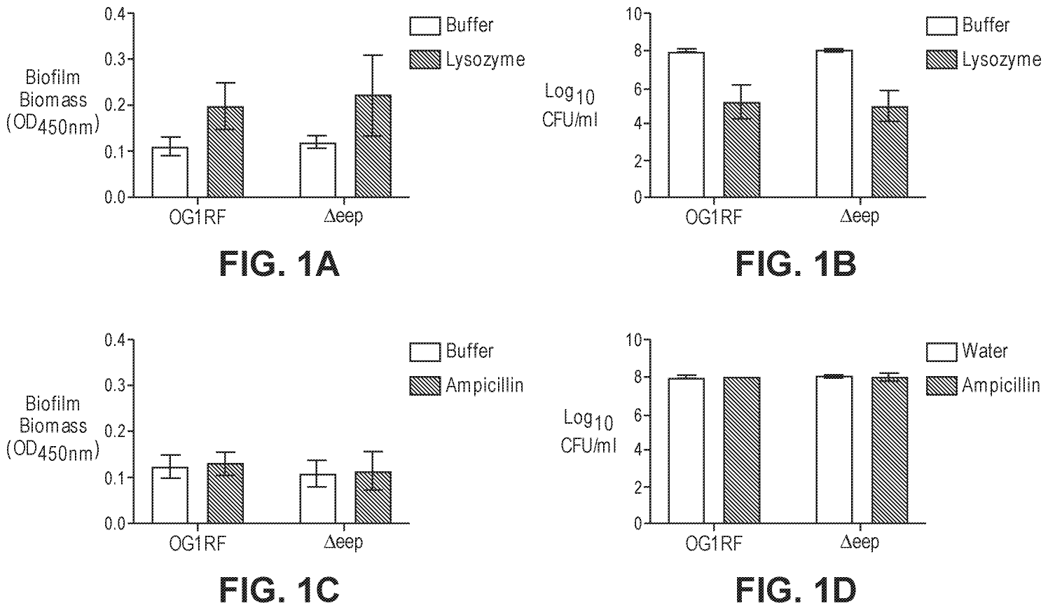

Biofilms of the two strains were grown overnight in tryptic soy broth without added glucose in the wells of 96-well polystyrene plates at 37° C. The liquid cultures were removed from the plate, and the material remaining in the wells (i.e., the biomass) was washed five times with sterile water. A lysozyme solution was prepared by dissolving 5 mg/ml hen egg white lysozyme in 10 mM Tris-HCl pH 8. Aliquots of lysozyme solution or buffer were added on top of the biofilm biomass of both strains in the 96-well plate, and the plate was incubated at 37° C. for three hours. Following incubation, the lysozyme or buffer solutions were removed, and the wells were washed again as above. The plates were allowed to dry for several hours, and then the biomass was stained with safranin, a non-specific stain that binds to bacterial cells and the biofilm matrix. Excess safranin was washed away, and the plates were dried again. Biofilm biomass was quantified by reading the optical density of safranin-stained wells at OD_{450 nm}. The results of this experiment are shown in FIG. 1A. It was observed that the biofilm biomass increased equally for both strains, which was unexpected.

Following this unexpected observation, the number of viable cells that could be recovered from the biofilm biomass following the three-hour treatment with lysozyme or buffer were measured. Cells were scraped up from the wells of the 96-well plate following the post-treatment washing step, as described above. The scraped up cells were serially diluted and plated to assess the number of colony forming units (CFU) recovered per ml (CFU/ml). The results of this experiment are shown in FIG. 1B. Reproducible drops of 2.8 log ₁₀ CFU/ml (calculated as the difference in the means of 3 biological replicates) for OG1RF and 3 log ₁₀ CFU/ml for Δeep were observed. These drops represent 99.8% and 99.9% decreases in viable E. faecalis biofilm cells for the two strains, respectively, following three-hour exposure to 5 mg/ml lysozyme. The results in FIGS. 1A and 1B also indicate that the bactericidal effect of lysozyme observed is independent of Eep protease.

It was found that the safranin staining in the lysozyme-treated wells post-sterile water washing was unexpectedly greater than the staining in untreated wells; however, the corresponding lysozyme-treated strains showed fewer living cells than the untreated strains on the cell viability assay for all lysozyme concentrations tested. Therefore, it is concluded that the difference in biofilm stain densities between E. faecalis was due to lysozyme weakening the cell wall of the organism and the cells lysing, thereby allowing the released cell contents, such as DNA, to be stained by the safranin.

### Example 2—Ampicillin Treatment

Ampicillin was tested to determine if it caused a similar effect. Ampicillin is a beta-lactam antibiotic that targets the cell wall of actively dividing cells. The strain tested, E. faecalis OG1RF, is susceptible to ampicillin in planktonic conditions, but its biofilms are resistant to >128 μg/ml ampicillin (Frank et al. (2015), “Evaluation of the Enterococcus faecalis biofilm-associated virulence factors AhrC and Eep in rat foreign body osteomyelitis and in vitro biofilm-associated antimicrobial resistance,” PLoS One, 10:e0130187). The ampicillin exposure experiments were carried out as described above for lysozyme, except that the biofilm biomass was exposed to water or a solution of 128 μg/ml ampicillin prepared in water. As shown in FIGS. 1C and 1D, the biofilms were resistant to any effect by ampicillin.

### Example 3—Lysozyme Treatment: Varied Duration

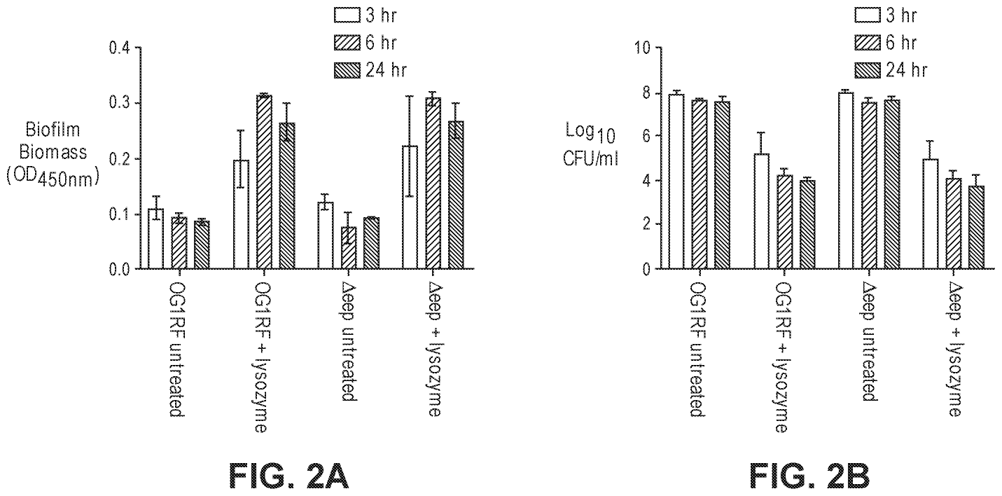

The effect of treating E. faecalis biofilms with 5 mg/ml lysozyme for 3 hours, 6 hours, and 24 hours at 37° C. was tested. The experiments were carried out as described above, except the time of lysozyme or buffer (labeled as “untreated” in FIGS. 2A and 2B) was varied. The results are shown in FIGS. 2A and 2B. The amount of stained biomass increased slightly at 6 hours compared to 3 hours. The number of viable cells recovered from the biofilm dropped slightly at 6 and 24 hours compared to 3 hours. Overall, it was concluded that only a nominal amount of extra killing of E. faecalis occurred after 3 hours when biofilms are exposed to 5 mg/ml lysozyme.

### Example 4—Lysozyme Treatment: Varied Concentration

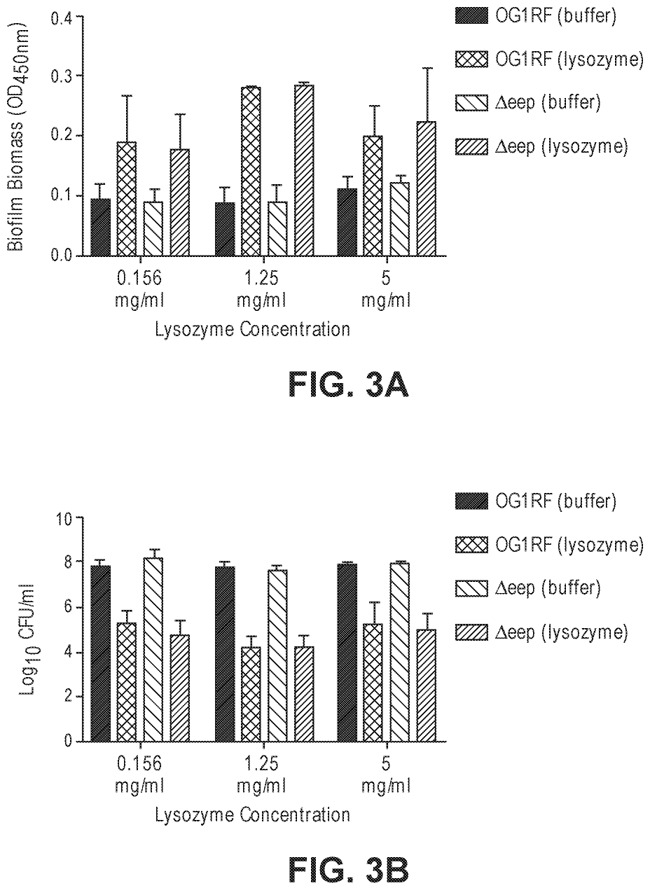

The range of lysozyme concentrations at which E. faecalis biofilm biomass was decreased was analyzed. The experiments were carried out as described above, except that concentrations of lysozyme of 0.156, 1.25, and 5 mg/ml were tested. The results are shown in FIGS. 3A and 3B. The largest increase in stained biomass correlated with the largest decrease in viable cells recovered, which occurred with 1.25 mg/ml lysozyme. The decrease in viable cells at 1.25 mg/ml lysozyme is about 3.5 log ₁₀ CFU/ml, which is a decrease of >99.9%.

### Example 5—Recombinant Human Lysozyme Treatment

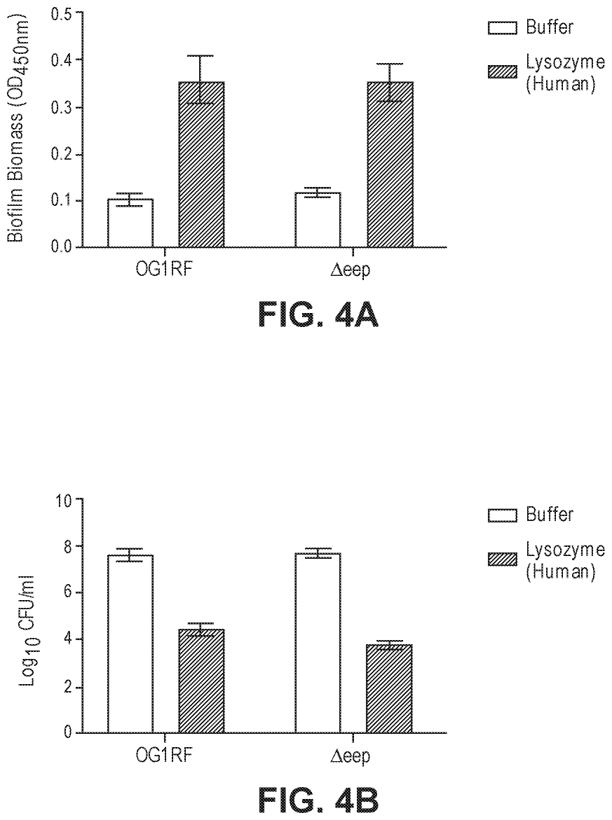

All of the experiments described above were conducted with hen egg white lysozyme. Purified, recombinant human lysozyme is available for purchase. Human lysozyme was tested to determine if it had a similar killing effect on E. faecalis biofilms. The results are shown in FIGS. 4A and 4B. The amount of stained biomass increased by approximately 3.5-fold. Human lysozyme reduced the number of viable E. faecalis cells recovered from OG1RF biofilms by 3.2 log ₁₀ CFU/ml and Δeep biofilms by 4.0 log ₁₀ CFU/ml, which both represent decreases of >99.9%.

### Example 6—Cellular Viability Assessment

Biofilms were grown overnight on Aclar discs, and non-adherent cells were washed away. The biofilms were then treated with 5 mg/ml hen egg white lysozyme for 3 hours at 37° C., and non-adherent cells were again washed away. The remaining biomass was stained with the LIVE/DEAD BacLight Bacterial Viability kit reagents (ThermoScientific) according to the manufacturer's instructions. Images were captured of stained biofilms obtained by fluorescence confocal microscopy from cultures of two E. faecalis strains (OG1RF and Δeep strains) after being treated with a lysozyme (hen egg white lysozyme) solution or a buffer solution lacking lysozyme. It was demonstrated that the amount of red-stained cells (indicating dead cells) sharply increased in the lysozyme-treated samples. The stained biofilm images corroborate the previous examples showing that lysozyme treatment of E. faecalis biofilms reduces bacterial viability.

### Example 7—DNA Assessment

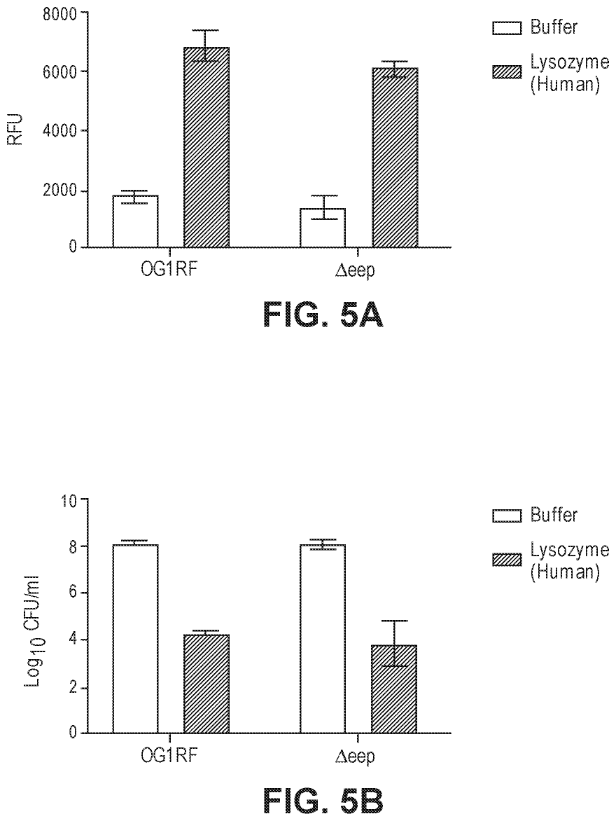

Taken together, the results above suggest that lysozyme lyses the E. faecalis cells. The increased biomass staining may be due to the release of DNA from the lysed cells. Quant-iT PicoGreen dsDNA Reagent (ThermoScientific) was used to measure the amount of DNA in E. faecalis biofilms post-treatment with lysozyme or buffer. Biofilms were formed in the wells of 96-well plates, and the assays were carried out as described above. Following the washing step after lysozyme or buffer alone treatment, a PicoGreen solution was added into the wells. The fluorescence was measured on a plate reader. FIGS. 5A and 5B show the relative amount of fluorescence (in relative fluorescence units, or RFU) and the corresponding number of viable bacteria recovered. The amount of DNA in the lysozyme-treated wells was approximately 3-fold higher than buffer-only wells.

### Example 8—Effect of Lysozyme on E. faecalis Biofilms: Wet and Dry Treatments

Both OG1RF and Δeep were streaked on Brain Heart Infusion (BHI) agar plates and incubated at room temperature for two days. Three colonies from each strain were inoculated in BHI broth and incubated overnight at 37° C. 96-well microtiter plate biofilm assays were performed using the overnight cultures diluted in Tryptic Soy Broth (TSB). Biofilm plates were incubated overnight at 37° C. in a moist environment. The microtiter plate was washed with sterile water. The effect of allowing the microtiter plate to dry before the addition of lysozyme treatment was examined. Three replicates of treating the biofilms in the microtiter plate with lysozyme immediately after washing, labeled as “wet”, and three replicates of treating the biofilms in the microtiter plate with lysozyme after allowing the plate to dry after washing, labeled as “dry”, were performed. Once the lysozyme was added, the plates were incubated for three hours. After incubation, the microtiter plate was washed again with sterile water and left to dry until water was absent in the wells. The plate was stained with safranin, washed again with sterile water, and then read on a plate reader to determine the amount of biofilm in the wells.

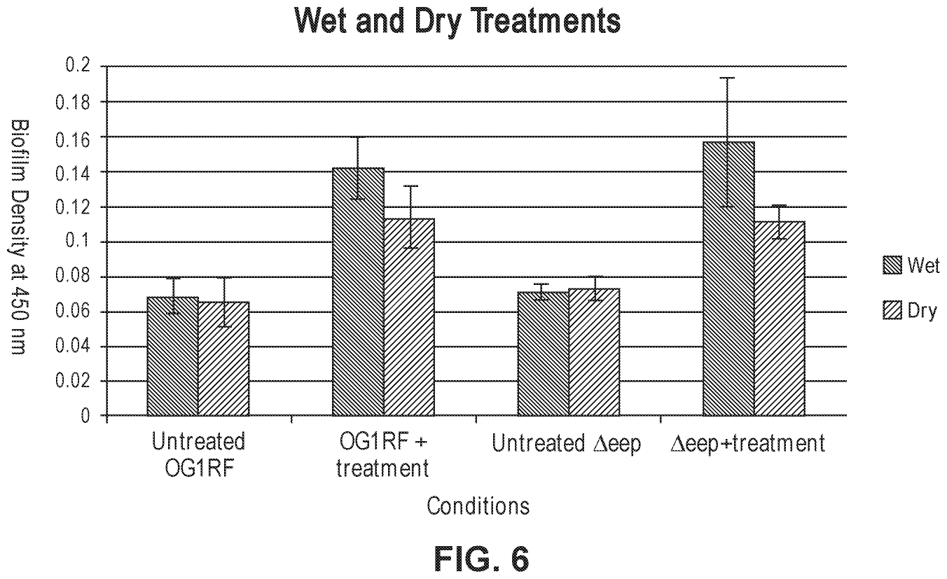

Both “wet” and “dry” assays resulted in similar data. It was concluded that hydration did not have a significant effect on E. faecalis' biofilm interaction with lysozyme. As illustrated in FIG. 6, for example, both untreated OG1RF and untreated Δeep have less biofilm staining density than treated OG1RF and treated Δeep. This result was unexpected, because the treated strains were expected to have less staining density because lysozyme would have a negative effect on E. faecalis.

While the data in FIG. 6 show an increase in biofilm staining density for both treated OG1RF and Δeep, cell viability assays showed that lysozyme did kill both strains of OG1RF and Δeep that were taken from the biofilm assay. These cell viability assays were done by scraping the biofilm of the strain from each of two conditions: one that was not treated with lysozyme, and one treated with lysozyme at a concentration of 5 mg/ml. The bacteria were then diluted (10⁰-10⁻⁷), and plated on Brain Heart Infusion (BHI) plates. The plates were incubated overnight at 37° C. Both treated strains displayed killing.

### Example 8—Lysozyme Treatment of E. faecalis OG1RF and Δeep Biofilms

It was next sought to confirm that the decrease observed in the number of live bacteria in a biofilm following lysozyme treatment was due primarily to loss of biofilm cell viability, and not merely dispersal of viable cells from the biofilm surface. To conduct this experiment, the number of viable cells that were present in the buffer or lysozyme solution that was removed from the biofilms following exposure was measured. If viable cells were dispersed from the biofilm following treatment, then one should be able to enumerate viable cells in the lysozyme solution removed from the treated wells.

In this experiment, biofilms of E. faecalis OG1RF and Δeep were grown in 96-well microtiter plates overnight. The biofilms were washed to remove non-adherent cells. Next, either buffer (10 mM Tris-HCl pH 8) or 5 mg/ml hen egg white lysozyme (in 10 nM Tris-HCl pH 8) was added to the wells, and the microtiter placed was incubated for 3 hours at 37° C.

Following incubation, the lysozyme and buffer solutions from two wells of OG1RF biofilms and two wells of Δeep biofilms were pipetted off. These solutions were diluted by serial 10-fold dilutions, and then aliquots of each dilution were plated on BHI agar plates to enumerate the number of viable bacteria present in each solution. In addition, the biofilms in the plate were washed, and then two wells of OG1RF biofilms and two wells of Δeep biofilms were removed by scraping manually with a pipette tip and resuspended in potassium phosphate buffered saline. These dislodged biofilm cell solutions were also diluted by serial 10-fold dilutions, and then aliquots of each dilution were plated on BHI agar plates to enumerate the viable bacteria present in each sample. Finally, the remainder of the biofilm wells were allowed to dry for several hours, and then the biomass in each well was stained with safranin. Excess safranin was washed away, and the plates were dried again. Biofilm biomass was quantified by reading the optical density of safranin-stained wells at OD₄₅₀ nm.

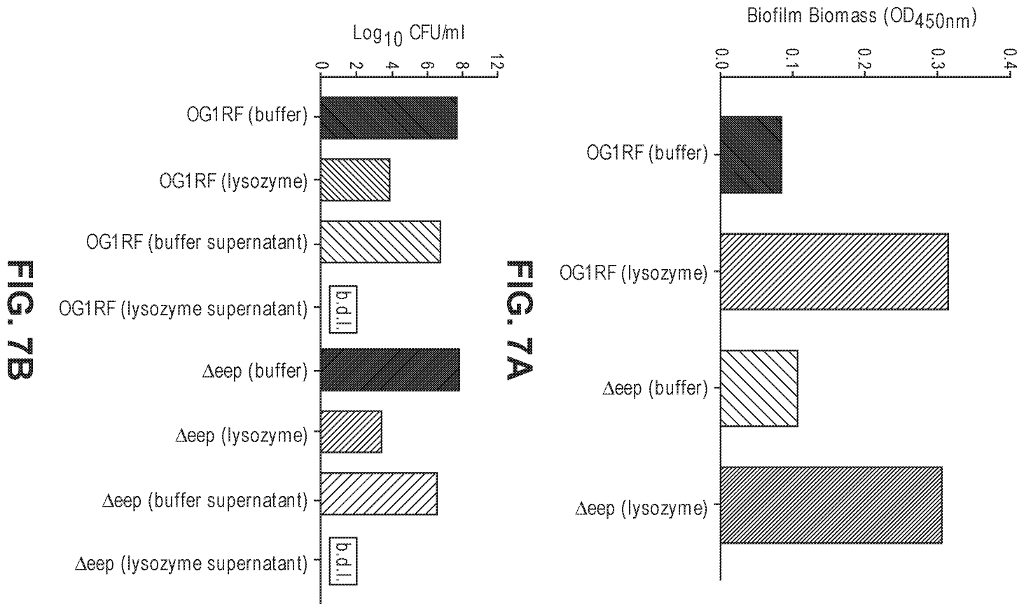

FIG. 7A shows the resulting optical densities of the stained biofilm biomasses. As shown in FIG. 7A, the biomasses of both the stained E. faecalis OG1RF and Δeep incubated in buffer solution were significantly less than the biomasses of E. faecalis OG1RF and Δeep incubated in lysozyme solution.

The quantity of viable biofilm cells was calculated as Log 10 CFU/mL, and the results indicate that lysozyme treatment decreased the number of viable biofilm cells, rather than merely dispersing viable cells from the biofilm. As shown in FIG. 7B, the quantity of viable OG1RF and Δeep cells from dislodged biofilms treated with buffer was greater than the quantity of viable cells from dislodged biofilms treated with lysozyme solution. FIG. 7B also illustrates that the number of viable cells recovered from the buffer (i.e., “buffer supernatant”) was greater than the number of viable cells recovered from the lysozyme solution (i.e., “lysozyme supernatant”). The data confirm that lysozyme treatment leads to loss of viability of biofilm cells, and not dispersal of viable cells from the biofilm. Specifically, high numbers of viable cells were enumerated in the “buffer supernatant” condition for each strain. In contrast, no viable cells above the limit of detection (illustrated in FIG. 7B as b.d.l., below detection limit) were measured in the “lysozyme supernatant condition” for either strain.

### Example 9—Lysozyme Exerts a Growth-Phase Dependent Reduction in Cell Viability on Planktonic E. faecalis Cells

It was hypothesized that the bactericidal effect of lysozyme on E. faecalis biofilm cells may be due, at least in part, to their growth phase. To test this hypothesis, E. faecalis survival in both water and lysozyme-containing broth medium was assessed for both logarithmic and stationary phase cells. Early logarithmic cultures were prepared by diluting overnight cultures 1:100 in BHI broth. To obtain stationary phase cells, BHI broth was inoculated with 3 individual colonies and cultivated for about 18 hours. Hen egg white lysozyme dissolved in sterile water was added to logarithmic and stationary phase cultures to a final concentration of 2.5 mg/ml. Cultures to which sterile water was added served as controls. Cultures were incubated at 37° C. for 6 hours. In order to determine the number of viable bacterial cells, aliquots of each culture were serially diluted and plated onto BHI agar at 0 and 6 hours post-exposure to lysozyme.

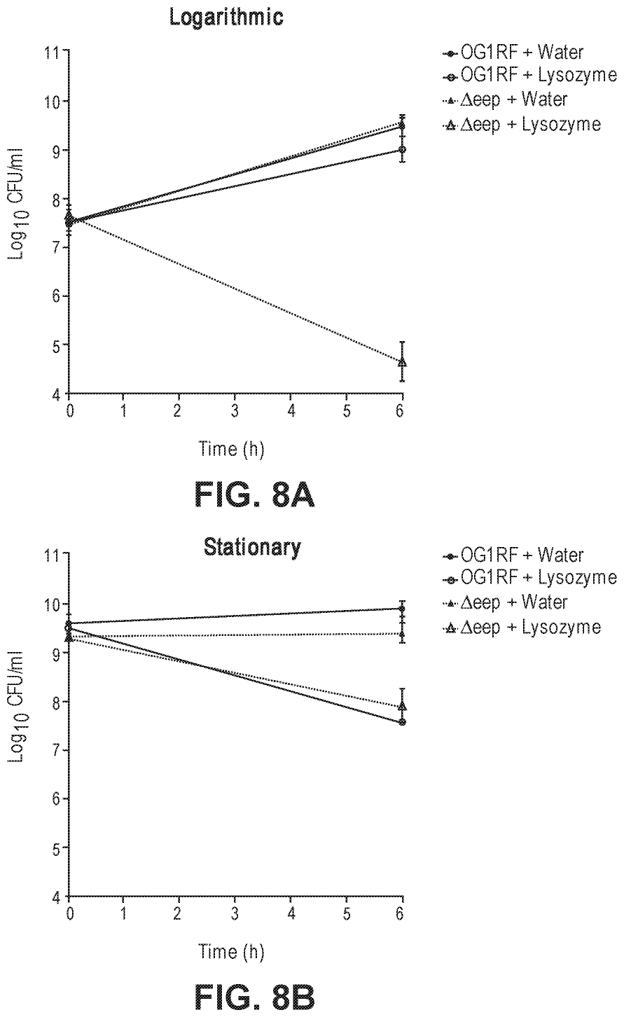

FIG. 8A shows that the number of viable Δeep cells following exposure to lysozyme for 6 hours during logarithmic growth decreased by about 3 log ₁₀ CFU/ml, whereas there is no decrease noted for OG1RF cells under the same experimental conditions. In contrast, FIG. 8B shows that the number of viable cells of OG1RF and Δeep decreases equally, by about 1.5 log ₁₀ CFU/ml, when planktonic cells in stationary phase are exposed to lysozyme for 6 hours. The reduction in viable OG1RF and Δeep caused by lysozyme in the stationary phase planktonic cells was similar to the effects observed following lysozyme treatment of biofilm cells.

### Example 10—Viability of E. faecalis Laboratory Strains and Clinical Isolates Reduced in Biofilms Following Exposure to Lysozyme

The ability of lysozyme to reduce the number of viable biofilm cells of other strains of E. faecalis in addition to OG1RF and Δeep was assessed. E. faecalis strains DS16, FA2-2, JH2-2, and 39-5 are strains that have been used for laboratory experiments for many years, and E. faecalis strain V583 is a vancomycin-resistant strain that has become the prototype lab strain for studies of vancomycin-resistant E. faecalis. E. faecalis strain VA1128 is a clinical isolate.

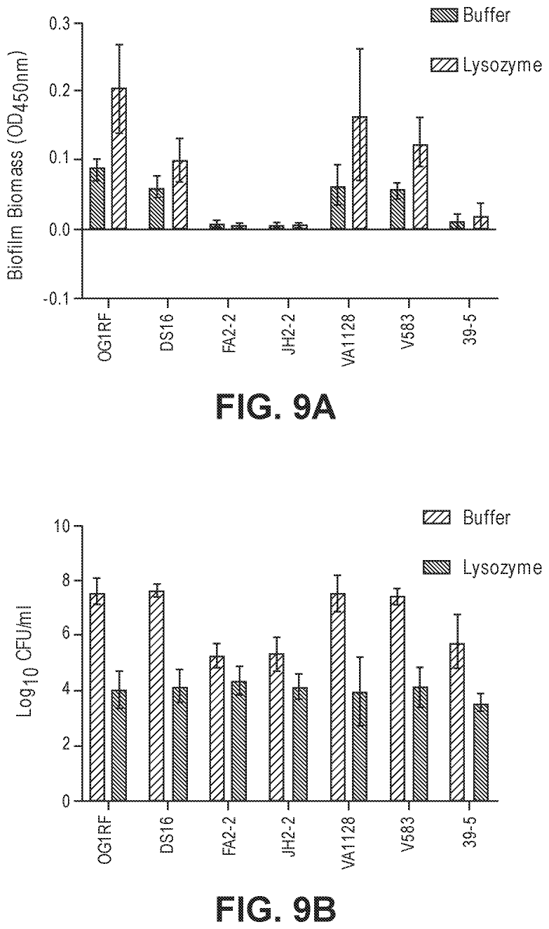

The same methods described above in Example 1 for assays evaluating lysozyme activity against E. faecalis OG1RF and Δeep biofilms were followed. FIG. 9A shows that biofilm biomass increased, to some extent, after lysozyme treatment for all the strains that made the most prominent amount of biofilm biomass (i.e., DS16, VA1128, and V583). Strains FA2-2, JH2-2, and 39-5 did not make prominent amounts of biofilm biomass. FIG. 9B shows that treatment of biofilms with lysozyme reduced the number of viable cells recovered from biofilms of all tested strains, including the three strains that did not make prominent amounts of biomass. These data confirm that the ability of lysozyme to reduce viable E. faecalis cells from biofilms is generalizable across multiple E. faecalis strains, including those that are vancomycin resistant and those that are clinical isolates.

All patents, patent applications, and published references cited herein are hereby incorporated by reference in their entirety. While this invention has been particularly shown and described with references to exemplary embodiments thereof, it will be understood by those skilled in the art that various changes in form and details may be made therein without departing from the scope of the invention encompassed by the appended claims.

1. A method of treating a bacterial infection associated with a biofilm, the method comprising administering a therapeutically effective amount of lysozyme to a subject that is infected with bacteria that produce the biofilm in and/or on the subject, wherein the lysozyme is exogenous to the subject.

2. The method of claim 1, wherein an etiologic agent of the bacterial infection is Enterococcus faecalis.

3. The method of claim 1, wherein the lysozyme is from chicken egg white.

4. The method of claim 1, wherein the lysozyme is recombinant human lysozyme.

5. The method of claim 1, wherein the subject is a mammalian subject.

6. The method of claim 1, further comprising administering a therapeutically effective amount of an antibacterial agent or a pharmaceutically acceptable salt thereof to the subject.

7. The method of claim 1, comprising topically administering the therapeutically effective amount of the lysozyme to the subject.

8. The method of claim 1, comprising administering the therapeutically effective amount of the lysozyme to an eye of the subject.

9. The method of claim 1, wherein the biofilm is on a medical device or exogenous biological component implanted in the subject.

10. The method of claim 1, comprising administering the therapeutically effective amount of the lysozyme in a solution that comprises a concentration of the lysozyme between about 0.1 mg/ml and about 10.0 mg/ml.

11. The method of claim 1, comprising administering the therapeutically effective amount of the lysozyme to the subject for between about three hours and about 24 hours.

12. A method of monitoring bacterial growth, the method comprising:
contacting a sample comprising a population of target bacterial organisms that produces a biofilm with a solution that comprises a concentration of lysozyme between about 0.1 mg/ml and about 10.0 mg/ml for between about three hours and about 24 hours; and,
detecting at least one property of the population of target bacterial organisms indicative of bacterial growth prior to, during, and/or after the contacting step, thereby monitoring the bacterial growth.

13. The method of claim 12, wherein the population of target bacterial organisms comprises Enterococcus faecalis.

14. The method of claim 12, wherein the property comprises an amount of biomass in the population of target bacterial organisms in the sample.

15. The method of claim 12, wherein the sample is from a mammalian subject.

16. A kit, comprising: a medical device that contains a solution that comprises an antibacterial concentration of lysozyme or a medical device or an exogenous biological component and a container comprising a solution that comprises an antibacterial concentration of lysozyme.

17. The kit of claim 16, wherein the container comprises the medical device or the exogenous biological component.

18. (canceled)
